(19) United States
(12) Patent Application Publication
(10) Pub. No.: US 2008/0200544 A1
(43) Pub. Date: Aug. 21, 2008

(54) Oil agent and lubricant agent, moisturizer and external preparation composition containing the same

(75) Inventors: Kyoichi Takeda, Chiba (JP); Yuki Kokeguchi, Chiba (JP); Kiyotaka Kawai, Chiba (JP)

Correspondence Address: WOLF GREENFIELD & SACKS, P.C., 600 ATLANTIC AVENUE, BOSTON, MA, 02210-2206, US

(73) Assignee: Kokyu Alcohol Kogyo Co., Ltd., Narita-shi (JP)

(21) Appl. No.: 12/069,119

(22) Filed: Feb. 7, 2008

Related U.S. Application Data
(60) Provisional application No. 60/900,510, filed on Feb. 9, 2007.

(51) Int. Cl. A61K 8/37; C07C 69/732; A61Q 19/00

(52) U.S. Cl. 514/547; 560/181

(57) ABSTRACT

The present invention provides an oil agent having water solubility and oil solubility, as well as suppressed stickiness when applied to skin to exhibit excellent feeling of use, and also provides a lubricating agent, a moisturizing agent and an external preparation containing said oil agent.

The oil agent of the present invention comprises a dibasic acid ester compound of general formula I:

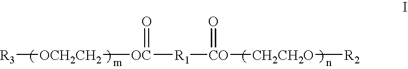

wherein R₁ is an alkylene group having a carbon number of 2-4, which may be mono- or poly substituted, R₂ and R₃ are, independently of each other, an alkyl group having a carbon number of 1-4, which may be mono- or poly substituted, n and m are, independently of each other, an integer of 0-4 with m+n≧1 (provided that the cases wherein both of R₂ and R₃ are an ethyl group and both of n and m are 1 are excluded).

### RELATED APPLICATION

This application claims the benefit of U.S. Provisional Application No. 60/900,510, filed Feb. 9, 2007, under 35 U.S.C. 119(e).

### FIELD OF THE INVENTION

The present invention relates to an oil agent having superior water solubility and oil solubility as well as suppressed stickiness when applied to skin, and to a lubricating agent, a moisturizing agent and an external preparation containing said oil agent.

### BACKGROUND ART

While there is a large demand for water-soluble oil agents, conventionally-known water-soluble or water-containing oil agents have a high viscosity so that they have some problems in the feeling of use when applied to skin. For example, glycerin is used in moisturizing agents, etc. to prevent the evaporation of water from the skin by forming a film on the skin surface; however, while it is necessary to increase the amount of blending the glycerin in order to obtain moist feeling when applied to the skin, when the amount of glycerin blending is sufficiently increased to obtain moist feeling, the feeling of oiliness also increases to generate stickiness, and therefore good feeling of use cannot be obtained.

Under such circumstance, with the aim of increasing moisturizing effects and suppressing stickiness, an external preparation such as a cosmetic material which contains an oligomer ester synthesized from divalent carboxylic acid and polyglycerin (Journal of Technical Disclosure of Japan Institute of Invention and Innovation (JIII) No. 2006-501820, Journal of Technical Disclosure of Japan Institute of Invention and Innovation (JIII) No. 2006-504729), and a lubricating agent having water solubility, lubrication and anti-rusting characteristics which contains the above oligomer ester (Journal of Technical Disclosure of Japan Institute of Invention and Innovation (JIII) No. 2006-505668) have been proposed. However, because these oligomer esters have high molecular weights and high viscosities, they can provide moisturizing effects but cannot sufficiently prevent stickiness when used as a cosmetic material.

A shampoo containing a plant-oil-derived water-soluble ester, a cation polymer, a pseudo-kaolinic polyamine, and a surfactant has been proposed with the aim of increasing the adsorption of treatment components that are applied to the hair after shampooing (JP, A, 2006-347972). Here, as the plant-oil-derived water-soluble ester, polyethylene glycol esters of plant oils such as jojoba oil, sunflower oil, macadamia ternifolia seed oil, olive oil, almond oil and castor oil have been listed; however, such esters derived from plant oils show stickiness when applied to skin, and therefore satisfactory good feeling of use cannot be obtained.

Furthermore, in general, water-soluble oil agents are hardly soluble in oil, and oil-soluble oil agents are hardly soluble in water. Oil agents that satisfy both of these features are not known. Therefore, in order to achieve a wide range of application of oil agents, an oil agent which is soluble in both water and oil, and shows no stickiness when applied to skin, has been strongly desired.

### DISCLOSURE OF THE INVENTION

### Problem to be Solved by the Invention

Therefore, an object of the present invention is to provide an oil agent having solubility in water and in oil which offers excellent feeling of use without stickiness when applied to skin, as well as a lubricating agent, moisturizing agent and external preparation containing said oil agent.

### Means of Solving the Problem

As a result of an intensive investigation taking into account the above-mentioned object, the present inventors have found that an ester consisting of a specific dibasic acid and a poly- (or mono-) ethylene glycol monoether has excellent solubility in water and in oil, and offers excellent feeling of use without stickiness when applied to skin, and the present invention has thus been accomplished.

Namely, the present invention relates to an oil agent comprising one or more dibasic acid ester compounds of general formula I:

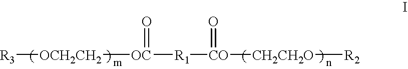

wherein R₁ is an alkylene group having a carbon number of 2-4, which may be mono- or poly substituted, R₂ and R₃ are, independently of each other, an alkyl group having a carbon number of 1-4, which may be mono- or poly substituted n and m are, independently of each other, an integer of 0-4 with m+n≧1 (provided that the cases wherein both of R₂ and R₃ are an ethyl group and both of n and m are 1 are excluded).

In addition, the present invention relates to said oil agent, wherein a poly- (or mono-)ethylene glycol monoether that constitutes the ester compound is one or two substances selected from the group consisting of diethylene glycol monoethyl ether, triethylene glycol monoethyl ether, diethylene glycol monopropyl ether, triethylene glycol monopropyl ether, diethylene glycol monoisopropyl ether and triethylene glycol monoisopropyl ether.

Furthermore, the present invention relates to said oil agent, wherein the dibasic acid is malic acid, succinic acid, tartaric acid or adipic acid.

In addition, the present invention relates to said oil agent, wherein the dibasic acid is succinic acid.

Furthermore, the present invention relates to said oil agent, wherein the solubility in water is 5% by mass or more at 20° C.

In addition, the present invention relates to said oil agent, wherein the viscosity is 5-200 (mPa·s) at 25° C.

Furthermore, the present invention relates to a lubricating agent comprising said oil agent.

In addition, the present invention relates to a moisturizing agent comprising said oil agent.

Furthermore, the present invention relates to an external preparation comprising said oil agent.

In addition, the present invention relates to said external preparation, wherein it is a cosmetic material.

The oil agent of the present invention contains an ester compound of general formula I which consists of a specific dibasic acid and a specific polyethylene glycol monoether, as mentioned above. Such an ester compound has a low viscosity and good solubility in water; therefore, by blending the oil agent into moisturizing lotions (cosmetic solution), hair-care products (in particular shampoos and hair mists, etc.) and make-up cosmetic materials (lip-care products and foundations, etc.), moisturizing agents and external preparations including cosmetic materials which offer appropriate tightness and moist feeling to the skin without stickiness can be provided. In addition, because the inventive oil agent has good solubility in water and in oil, it has a wide range of applicability, so that it can be applied to various fields such as in lubricating agents, paints and inks, etc.

### BEST MODE FOR CARRYING OUT THE INVENTION

### [1] Oil Agent

The oil agent of the present invention comprises one or more dibasic acid ester compounds of general formula I:

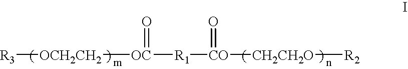

wherein R₁ is an alkylene group having a carbon number of 2-4, which may be mono- or poly substituted, R₂ and R₃ are, independently of each other, an alkyl group having a carbon number of 1-4, preferably a carbon number of 1-3, which may be mono- or poly substituted, and n and m are, independently of each other, an integer of 0-4, preferably 0-3, and more preferably 2-3 (provided that m+n≧1).

Examples of R₁ include alkylene groups such as ethylene, propylene, isopropylene, butylene and isobutylene; examples of substituents bound to the alkylene group include hydroxyl group, alkoxy group, substituted or non-substituted amino group, and ester group, etc.

The dibasic acid constituting the ester compound of general formula I is a dibasic acid with a carbon number of 4-6, and its specific examples include succinic acid, glutaric acid, adipic acid, malic acid and tartaric acid, etc. Preferably it is malic acid, succinic acid, tartaric acid or adipic acid. The dibasic acid is most preferably succinic acid.

Examples of R₂ and R₃ include alkyl groups with a carbon number of 1-4 such as methyl group, ethyl group, n-propyl group, isopropyl group, n-butyl group, isobutyl group, sec-butyl group and tert-butyl group; preferably they are an alkyl group with a carbon number of 1-3 such as methyl group, ethyl group, n-propyl group and isopropyl group. Examples of substituents bound to the alkyl group include hydroxyl group, alkoxy group, substituted or non-substituted amino group, and ester group, etc. The alkyl groups represented by R₂ and R₃ may be identical to or different from each other.

Specific examples of a poly- (or mono-)ethylene glycol monoether constituting the ester compound of general formula I include ethylene glycol monomethyl ether, ethylene glycol monoethyl ether, ethylene glycol monopropyl ether, ethylene glycol monoisopropyl ether, ethylene glycol monobutyl ether, ethylene glycol monoisobutyl ether, diethylene glycol monomethyl ether, diethylene glycol monoethyl ether, diethylene glycol monopropyl ether, diethylene glycol monoisopropyl ether, diethylene glycol monobutyl ether, diethylene glycol monoisobutyl ether, triethylene glycol monomethyl ether, triethylene glycol monoethyl ether, triethylene glycol monopropyl ether, triethylene glycol monoisopropyl ether, triethylene glycol monobutyl ether, triethylene glycol monoisobutyl ether, tetraethylene glycol monomethyl ether, tetraethylene glycol monoethyl ether, tetraethylene glycol monopropyl ether, tetraethylene glycol monoisopropyl ether, tetraethylene glycol monobutyl ether, tetraethylene glycol monoisobutyl ether, etc.; particularly preferred are diethylene glycol monoethyl ether, triethylene glycol monoethyl ether, diethylene glycol monopropyl ether, triethylene glycol monopropyl ether, diethylene glycol monoisopropyl ether, triethylene glycol monoisopropyl ether, etc.

The above dibasic acid together with a same kind of poly-(or mono-)ethylene glycol monoethers, or together with two different kinds of poly- (or mono-)ethylene glycol monoethers, may form an ester compound. However, cases wherein R₂ and R₃ are both an ethyl group, and n and m are both 1, are excluded.

In the oil agent of the present invention, only one kind of the ester compounds of the above general formula I may be used, or two or more kinds of said ester compounds may be used together.

The inventive oil agent has a high solubility in water. The water solubility of the inventive oil agent at 20° C. is preferably 5% by mass or more, more preferably 10% by mass or more, and furthermore preferably the oil agent can be dissolved in water at any ratio.

The inventive oil agent also has a high solubility in oil in addition to water. For example, the solubility in KAK99 (isononyl isononanoate) of the inventive oil agent at 20° C. is preferably 5% by mass or more, more preferably 10% by mass or more, and furthermore preferably the oil agent can be dissolved in KAK99 at any ratio.

The viscosity of the oil agent of the present invention is, when used in external preparations such as moisturizers and cosmetic materials, preferably 5-200 (mPa·s, 25° C.), and more preferably 8-100 (mPa·s, 25° C.). When the viscosity is within the above range, good feeling of use without stickiness can be obtained upon application to skin, etc. In addition, when the oil agent is used in lubricating agents, the viscosity is preferably 50-200 (mPa·s, 25° C.), and more preferably 60-180 (mPa·s, 25° C.). When the viscosity is within this range, appropriate lubricating property and workability can be obtained. In this specification, “viscosity” refers to a value of viscosity measured using a Brookfield viscometer DV-II+ (Spindle No. 2, 12 rpm, 25° C.).

The acid value of the oil agent of the present invention is preferably 3 or less, and more preferably 1 or less. When an oil agent has a high acid value, it causes odor and irritation in the skin; accordingly, a low acid value is preferred. The hydroxyl value of the oil agent is preferably 300 or less, and more preferably 150 or less. When a hydroxyl value is within this range, the viscosity becomes low, leading to refreshing feeling of use and water solubility of the oil agent.

The color (APHA) is desirably low for external preparations such as moisturizers and cosmetic materials, and is preferably 50 or less, and more preferably 20 or less.

### [2] Process for Producing Oil Agent

A process for producing the oil agent of the present invention is not particularly limited, and may employ a well-known method, or an appropriate combination of well-known methods. In general, the inventive oil agent can be synthesized by the ester condensation of a dibasic acid and a poly- (or mono-)ethylene glycol monoether. The reaction temperature of the ester condensation is preferably 120-160° C. Examples of a catalyst include sodium hydroxide, p-toluenesulfonic acid, boron trifluoride, hydrogen fluoride, tin chloride, zinc, titanium, potassium hydroxide, mineral acid (sulfuric acid, hydrochloric acid, etc.), zinc chloride, hypophosphorous acid, and dibutyltin oxide etc., and it is preferable to use p-toluenesulfonic acid, hypophosphorous acid, etc. The reaction may also be carried out without a catalyst. Examples of a reaction solvent include benzene, toluene and xylene, etc., and it is preferable to use toluene, xylene, etc. The reaction may also be carried out without a solvent. The ester condensation reaction is carried out using an acid value as the indicator, and it is carried out until the acid value becomes preferably 3 or less, and more preferably 1 or less.

When an ester compound (cross-esterification product) is to be prepared from a dibasic acid and two different kinds of poly- (or mono-)ethylene glycol monoethers, it is possible to react, for example, a dibasic acid with two kinds of poly- (or mono-) ethylene glycol monoethers to produce a mixed-system ester compound. In general, cross-esterification product are obtained from a mixed-system ester compound at a yield of approximately 50%. Ester compounds can be used for the inventive oil agent as a mixed system, or they can be used after isolation.

### [3] External Preparation

The external preparation of the present invention is not particularly limited as long as it is an external preparation comprising the inventive oil agent and is applicable to skin, hair, mucosa, wounds, etc.; it includes various types of cosmetic materials, quasi drugs, medicinal drugs, etc.

Examples of the cosmetic materials include emulsions, creams (skin cream, lip cream, hair cream, etc.), liquid foundation, eyeliner, mascara, eye shadow gel, lipstick, lipgloss, eye gloss, eye color, blusher, body gloss, ointments, soaps, mousses, tonics, gels, shampoo, hair mist, bath agents, and nail care products.

Examples of the medicinal drugs and quasi drugs include preparations such as lotions, creams, ointments, sprays, aerosols, skin patches, and gels. Examples of active medicinal agents include anti-infective agents (antiviral agents, etc.), analgesics or analgesic mixtures, arthritis drugs, antidepressants, diabetes drugs, antihistamine agents, anti-inflammatory agents, migraine preparations, antiemetic agents, antitumor agents, antipruritics, psychosis drugs, xanthine derivatives, calcium channel blockers, beta blockers, antiarrhythmic agents, antihypertensive agents, diuretics, cardiovascular preparations, hormones, immunosuppressive drugs, muscle relaxants, vasoconstrictors, vasodilators, wound healing promoters, allergy inhibitors, anti-acne agents, antiaging agents, antitussive agents, hemorrhoid drugs, local anesthetics, inflammation inhibitors, and anticholinergic agents.

The external preparation of the present invention may contain, depending on the intended purpose or as necessary, a skin-whitening agent, a moisturizer, an antioxidant, an anti-inflammatory agent, vitamins, a hormonal agent, an enzyme, a circulation promotion agent, aminoacids, an UV-absorbing agent, a sunscreen agent, a suntan agent, a hair growth agent (a hair-loss prevention agent, a hair growth promotion agent, etc.), an animal or plant extract, an anti-wrinkle agent, an antiseptics, a hair softener, a hair moisturizer, a makeup preparation, a hair conditioner, a skin conditioner, a hair whitening agent, a chelating agent, a cell replacement promotion agent, a coloring agent, a skin softening agent or a skin moisturizer, a deodorant or an antiperspirant, etc.

Examples of the skin-whitening agent include hydroquinone derivatives [hydroquinone glycosides such as hydroquinone α-D-glucose, hydroquinone β-D-glucose (arbutin), hydroquinone α-L-glucose, hydroquinone β-L-glucose, hydroquinone α-D-galactose, hydroquinone β-D-galactose, hydroquinone α-L-galactose, and hydroquinone β-L-galactose, etc.], kojic acid and derivatives thereof, L-ascorbic acid and derivatives thereof [L-ascorbic acid monoesters such as L-ascorbic acid monophosphate and L-ascorbic acid 2-sulfate, L-ascorbic acid glucosides such as L-ascorbic acid 2-glucoside, and salts thereof], tranexamic acid or derivatives thereof [tranexamic acid dimer (trans-4-(trans-aminomethylcyclohexanecarbonyl)aminomethylcy clohexanecarboxylic acid hydrochloride, etc.), esters of tranexamic acid and hydroquinone (4′-hydroxyphenyl trans-4-aminomethylcyclohexanecarboxylate, etc.), esters of tranexamic acid and gentisic acid (2-(trans-4-aminomethylcyclohexylcarbonyloxy)-5-hydroxybenzoic acid and salts thereof, etc.), amides of tranexamic acid (trans-4-aminomethylcyclohexanecarboxylic acid methylamide and salts thereof, etc.)], ellagic acid and derivatives thereof, salicylic acid and derivatives thereof [3-methoxysalicylic acid and salts thereof, 4-methoxysalicylic acid and salts thereof, 5-methoxysalicylic acid and salts thereof, etc.], resorcinol derivatives [alkylresorcinols such as 4-n-butylresorcinol and salts thereof, etc.], and plant extracts having a skin-whitening action.

Examples of the anti-inflammatory agent include glycyrrhizic acid salts (dipotassium glycyrrhizinate, ammonium glycyrrhizinate, etc.), allantoin, and mixtures thereof.

Examples of the antimicrobial agent include resorcin, sulfur, salicylicacid, zincpyrithione, photosensitizer No. 101, photosensitizer No. 102, Octopirox, hinokitiol, bacitracin, erythromycin, neomycin, tetracycline, chlorotetracycline, benzethonium chloride, phenol, multivalent alcohols (1,2-pentanediol, 1,2-hexanediol, 1,2-octanediol, etc.) and mixtures thereof.

Examples of the vitamins include vitamins A, C, D, E, and K, vitamin A palmitate, thiamine, vitamin B₆, vitamin B₆ derivatives such as vitamin B₆ hydrochloride, vitamin B₂, vitamin B₁₂, nicotinic acid, nicotinic acid derivatives such as nicotinic-acidamide, pantothenic acid, pantothenyl ethyl ether, pyridoxine, inositol, vitamin B complex such as carnitine, panthenol, and mixtures thereof.

Examples of the hormonal agent include oxytocin, corticotropin, vasopressin, secretin, gastrin, and calcitonin.

Examples of the enzyme include trypsin, lysozyme chloride, chymotrypsin, chymotrypsin-like enzymes, aspartic proteinase, semialkali proteinase, serrapeptase, lipase, and hyaluronidase.

Examples of the antioxidant include thiotaurine, glutathione, catechin, albumin, ferritin, metallothionein, and the above-mentioned L-ascorbic acid and derivatives thereof.

Examples of blood circulation promoters include acetylcholine derivatives, cepharanthin, and carpronium chloride.

Examples of the amino acids include amphoteric amino acids such as stearyl acetyl glutamate, capryloyl silk amino acid, capryloyl collagen amino acid, capryloyl keratin amino acid, capryloyl p amino acid, cocodimonium hydroxypropyl silk amino acid, corn gluten amino acid, cysteine, glutamic acid, glycine, hair amino acids such as hair keratin amino acid and aspartic acid, threonine, serine, glutamic acid, proline, glycine, alanine, half-cystine, valine, methionine, isoleucine, leucine, tyrosine, phenylalanine, cysteic acid, histidine, arginine, cysteine, tryptophan, citrulline, lysine, silk amino acids, wheat amino acids, and mixtures thereof.

Examples of the UV-absorbing agent include benzophenone, bornelone, butyl PABA, cinnamidopropyl trimethyl ammonium chloride, disodium distyrylbiphenyl disulfonate, PABA, potassium methoxycinnamate, and mixtures thereof.

Examples of the sunscreen agent include butylmethoxydibenzoylmethane, octylmethoxycinnamate, octocrylene, octylsalicylate, phenylbenzimidazole sulfonic acid, ethyl hydroxypropylaminobenzoate, menthyl anthranilate, aminobenzoic acid, cinoxate, diethanolamine methoxycinnamate, glyceryl aminobenzoate, titanium dioxide, zinc dioxide, oxybenzone, Padimate-O, red petrolatum, and mixtures thereof.

Examples of the hair growth agent include blood circulation promoters such as swertia japonica extract, acetylcoline derivatives, cepharanthin and carpronium chloride, local stimulants such as red pepper tincture, cantharis extract and nonylic acid vanillamide, antiseborrhea agents such as pyridoxine and derivatives thereof, antimicrobial agents such as benzalkonium chloride, isopropylmethylphenol, zinc pyrithione, photosensitizer No. 101, photosensitizer No. 102, Octopirox and hinokitiol, metabolic stimulants such as photosensitizer No. 301, placenta extract and biotin, amino acids such as serine, methionine and tryptophan, and vitamins such as vitamins B₂ and B₁₂, pantothenic acid and derivatives thereof.

Among the animal and plant extracts, examples of the plant extract include tea extract, rosa roxburghii extract, scutellaria root extract, houttuynia cordata extract, phellodendri cortex extract, melilotus officinalis extract, lamium album extract, glycyrrhiza extract, paeoniae radix extract, saponaria officinalis extract, luffa cylindrica extract, cinchona extract, saxifrage extract, sophora angustifolia extract, nuphar haponicum root extract, fennel extract, primrose extract, rose extract, rehmannia extract, lemon extract, lithospermum root extract, aloe extract, iris root extract, eucalyptus extract, equisetum arvense extract, sage extract, thyme extract, seaweed extract, cucumber extract, clove extract, raspberry extract, melissa officinalis extract, carrot extract, horse chestnut extract, peach extract, peach leaf extract, mulberry extract, centaurea cyanus extract, witch hazel extract, ginkgo extract, wintergreen extract, swertia japonica extract, red pepper tincture extract, and cantharis extract. Examples of the animal extract include placenta extract and collagen.

The external preparation of the present invention may contain an oily component other than the oil agent of the present invention. As the other oily component, a component of any origin such as an animal oil, a plant oil, a synthetic oil, etc. and a component with any properties such as a solid oil, a semi-solid oil, a liquid oil, a volatile oil, etc. may be used, and examples thereof include hydrocarbons, silicone oils, fats, waxes, hydrogenated oil, ester oils, fatty acids, fatty alcohols, fluorine-containing oils, and lanolin derivatives. Specific examples thereof include hydrocarbons such as light liquid isoparaffin, liquid paraffin, squalane, vaseline, polyisobutylene and polybutene, oils such as olive oil, castor oil, jojoba oil, mink oil and macadamia ternifolia seed oil, petroleum waxes such as paraffin wax and microcrystalline wax, mineral waxes such as ozokerite and ceresine, natural waxes such as carnauba wax and candelilla wax, esters such as cetyl octanoate, isopropyl myristate, isopropyl palmitate, octyldodecyl myristate, glyceryl trioctanoate, polyglyceryl diisostearate, diglyceryl triisostearate, glyceryl tribehenate, pentaerythritol rosinate, neopentyl glycol dioctanoate and cholesterol fatty acid esters, fatty acids such as stearic acid, lauric acid, myristic acid, behenic acid, isostearic acid, oleic acid and 12-hydroxystearic acid, fatty alcohols such as stearyl alcohol, cetylalcohol, laurylalcohol, oleylalcohol, isostearyl alcohol and behenyl alcohol, silicones such as low-degree-of-polymerization dimethylpolysiloxane, decamethylcyclopentasiloxane, octamethylcyclosiloxane, high-degree-of-polymerization dimethylpolysiloxane, methylphenylpolysiloxane, polyether-modified polysiloxane, a polyoxyalkylene/alkylmethylpolysiloxane/methylpolysiloxane copolymer, alkoxy-modified polysiloxane and fluorine-modified polysiloxane, fluorine-containing oils such as perfluorodecane, perfluorooctane and perfluoropolyether, and lanolin derivatives such as lanolin, lanolin acetate, lanolin fatty acid isopropyl ester and lanolin alcohol.

The external preparation of the present invention may contain, in addition to the oil agent and the above-mentioned components, as appropriate and as necessary a component that is normally used in an external preparation, such as a moisturizer, a surfactant, a UV-absorbing agent, a UV-scattering agent, a fragrance, a viscosity increasing agent, an antiseptic, a coloring agent such as an extender pigment and a coloring pigment, a pH adjusting agent, and a chelating agent (citric acid, phosphoric acid, EDTA-4Na, etc.) in a range that does not impair the effects of the present invention.

The form of the external preparation of the present invention is not particularly limited; the external preparation may be in the form of a liquid, an emulsion, a semi-solid agent, a solid agent, etc. according to its intended purpose. The emulsion is not particularly limited and may be any of a water-in-oil type (W/O type) or an oil-in-water type (O/W type), a W/O/W type, and an O/W/O type, etc. For example, in the case of a water-in-oil type emulsion cosmetic material, it is prepared by adding water and a water-soluble component (aqueous phase component) to the oil agent. The content ratio of the oil agent and the aqueous phase component may be adjusted in accordance with desired properties of the emulsion, and is not particularly limited.

### [4] Moisturizing Agent

The moisturizing agent of the present invention may be used in, in addition to the above-mentioned external preparations, adhesives (stick-type glue, etc.), inks (ink-jet ink, aqueous ink, etc.), sanitary paper (lotion tissue paper, lotion toilet paper, etc.), moisturizing supporters, moisture-adjusting sheet, bed-bath sheet, etc. For example, when the moisturizing agent is used in an aqueous ink, the ink is prepared by adding the oil agent of the invention together with other components used in general inks such as an aqueous pigment, a surfactant, a chelating agent, a dissolution auxiliary agent, a penetration control agent, a viscosity adjusting agent, a pH adjusting agent and an antioxidant, into an aqueous solution. In addition, when the moisturizing agent of the invention is used in sanitary paper, the sanitary paper containing the moisturizing agent may be prepared by coating, impregnating, or spray coating the moisturizing agent onto a sheet.

The moisturizing agent of the invention is not limited so long as it contains the oil agent of the invention, and it may co-use other moisturizing agents within a range of not detracting the purpose of the invention. Examples of the other moisturizing agents include polyvalent alcohol moisturizing agents (glycerin, diglycerin, propylene glycol, dipropylene glycol, etc.), sugar-containing moisturizing agents (trehalose, sulfated tolehalose, pullulan, maltose, carboxymethyl dextran, etc.), amino-acid-containing moisturizing agents (proline, glycine, serine, threonine, glutamic acid, aspartic acid, lysine, etc.)

### [5] Lubricating Agent

The lubricating agent of the present invention can be used for, for example, a metal processing fluid which is used for cutting and grinding of metals, a hydraulic actuation fluid which is a pressure transmission medium in hydraulic systems, a lubricating fluid or an actuating fluid for action parts in machines and devices, etc., an anticorrosion fluid used upon processing of machines and devices, an anticorrosion lubricating fluid used for washing systems, a coolant liquid used for washing, grinding and molding of metals and quench annealing of steels.

The lubricating agent of the invention may appropriately blend other additives as requested, such as fatty acids (straight-chain saturated fatty acids, monoene unsaturated fatty acids, polyene unsaturated fatty acids, alicyclic fatty acids, monohydroxy fatty acids, dihydroxy fatty acids, etc.), fatty acid esters (an ester of the above fatty acid with an alkyl alcohol, etc.), anticorrosion agents (carboxylic acids such as naphthene acid, alkenyl succinate, etc., or metal salts (Na, K, Ca, Mg, etc.) thereof, etc.), surfactants (nonionic, anionic, cationic, or ampholytic surfactants), antiseptics (1,2-alkanediol, phenol, triazine, or isothiazoline antiseptics, etc.), antifoam agents (silicone emulsions, fatty alcohols, metal soaps, etc.).

### EXAMPLES

The present invention is explained in further detail by reference to Examples below, but the present invention should not be construed as being limited thereto.

In these examples, viscosity was measured using a Brookfield viscometer DV-II+ (Spindle No. 2, 12 rpm, 25° C.).

Acid values and hydroxyl values were measured in accordance with General Test Methods, 18: Acid Value Measurement Method and 24: Hydroxyl Value Measurement Method, Japanese Standards of Cosmetic Ingredients (newly revised, first edition, 30 Aug. 1999). In addition, color (APHA) was measured using OME2000 (Nippon Denshoku Industries, Co., Ltd.).

### Example 1

To a four-necked 1-L flask equipped with a stirring device, thermometer, reflux apparatus and nitrogen-gas supply nozzle, 177.2 g (1.5 mol) of succinic acid, 603.9 g (4.5 mol) of 2-(2-ethoxyethoxy)ethanol commonly known as carbitol (EDG; Yamaichi Chemical Industries, Co., Ltd.), 0.78 g of p-toluenesulfonic acid as a catalyst, and 120 ml of toluene as a solvent were added, and the reaction was carried out at 160° C. under reflux until the acid value became 3 or less (OH group/COOH group=1.5). The reaction time was approximately 15 h. Then, the reaction solution was decompressed to approximately 5 mmHg at 160° C., and unreacted carbitol was distilled away. To the reaction solution from which EDG was removed, 15.6 g of sodium carbonate decahydrate was added and stirred for 1 h at 120° C. This slurry reactant was filtered to obtain 497 g of pale yellow dicarbitol succinate. FIGS. 1 and 2 show IR and GC/MS charts of the obtained dicarbitol succinate. Table 1 shows measured results of the acid value, saponification value, hydroxyl value, color (APHA), viscosity, water solubility, oil solubility and feeling of use of the dicarbitol succinate.

TABLE 1

| | | | | | | | Comparative |
|---|---|---|---|---|---|---|---|
| | Example 1 | Example 2 | Example 3 | Example 4 | Example 5 | Example 6 | example 1 |
| Acid value | 0.2 | 0.6 | 0.8 | 0.9 | 0.4 | 0.3 | 0.6 |
| Saponification | 304 | 291 | 282 | 279 | 260 | 203 | 194 |
| value | | | | | | | |
| Hydroxyl | 0.9 | 147 | 1.2 | 264 | 1.1 | 0.8 | 1.5 |
| value | | | | | | | |
| Color | 30 | 62 | 45 | 50 | 60 | 54 | 65 |
| (APHA) | | | | | | | |
| Viscosity*¹ | 12.5 | 45 | 20 | 207 | 32.5 | 10.0 | 5 |
| Water | ⊚ | ◯ | ◯ | ⊚ | ⊚ | Δ | X |
| solubility*² | | | | | | | |
| Oil | ⊚ | Δ | ◯ | ◯ | Δ | ⊚ | ⊚ |
| solubility*³ | | | | | | | |
| Feeling of | light | slightly | light | slightly | slightly | light | light |
| use*⁴ | | light | | heavy | light | | |

*¹mPa · s, 25° C.
*²Evaluation: ⊚: Dissolved in water at an arbitrary ratio, ◯: Dissolved in water up to 10% by mass, Δ: Dissolved in water up to 5% by mass, X: Insoluble in water.
*³Evaluation: ⊚: Dissolved in KAK99 at an arbitrary ratio, ◯: Dissolved in KAK99 up to 10% by mass, Δ: Dissolved in KAK99 up to 5% by mass, X: Insoluble in KAK99.
*⁴Light: viscosity 5-20 (mPa · s, 25° C.) Slightly light: viscosity 21-50 (mPa · s, 25° C.) Moderate: viscosity 51-150 (mPa · s, 25° C.) Slightly heavy: viscosity 151-250 (mPa · s, 25° C.) Heavy: viscosity 251 (mPa · s, 25° C.) or more

### Example 2

The procedure of Example 1 was carried out except that 201.1 g (1.5 mol) of malic acid was used instead of succinic acid, to synthesize 505 g of dicarbitol malate. OH group/COOH group=1.5 (excluding OH groups in the malic acid), and the reaction time was 18 h. Table 1 shows measured results of the acid value, saponification value, hydroxyl value, color (APHA), viscosity, water solubility, oil solubility and feeling of use of the dicarbitol malate.

### Example 3

The procedure of Example 1 was carried out except that 219 g (1.5 mol) of adipic acid was used instead of succinic acid, to synthesize 539 g of dicarbitol adipate. OH group/COOH group=1.5, and the reaction time was 14 h. Table 1 shows measured results of the acid value, saponification value, hydroxyl value, color (APHA), viscosity, water solubility, oil solubility and feeling of use of the dicarbitol adipate.

### Example 4

The procedure of Example 1 was carried out except that 225 g (1.5 mol) of tartaric acid was used instead of succinic acid, to synthesize 545 g of dicarbitol tartrate. OH group/COOH group=1.5, and the reaction time was 16 h. Table 1 shows measured results of the acid value, saponification value, hydroxyl value, color (APHA), viscosity, water solubility, oil solubility and feeling of use of the dicarbitol tartrate.

### Example 5

The procedure of Example 1 was carried out except that 739 g (4.5 mol) of triethylene glycol monoethyl ether was used instead of carbitol, to synthesize 585 g of di(triethylene glycol monoethyl ether) succinate. OH group/COOH group=1.5, and the reaction time was 18 h. Table 1 shows measured results of the acid value, saponification value, hydroxyl value, color (APHA), viscosity, water solubility, oil solubility and feeling of use of the di(triethylene glycol monoethyl ether) succinate.

### Example 6

The procedure of Example 1 was carried out except that 406 g (4.5 mol) of monoethylene glycolmonoethyl ether was used instead of carbitol, to synthesize 374 g of di(monoethylene glycol monoethyl ether) succinate. OH group/COOH group=1.5, and the reaction time was 18 h. Table 1 shows measured results of the acid value, saponification value, hydroxyl value, color (APHA), viscosity, water solubility, oil solubility and feeling of use of the di(monoethylene glycol monoethyl ether) succinate.

### Comparative Example 1

The procedure of Example 1 was carried out except that 316 g (2 mol) of isononanoic acid was used instead of succinic acid and that 403 g (3 mol) of carbitol was used, to synthesize 521 g of dicarbitol isononanoate. OH group/COOH group=1.5, and the reaction time was 20 h. Table 1 shows measured results of the acid value, saponification value, hydroxyl value, color (APHA) viscosity, water solubility, oil solubility and feeling of use of the dicarbitol isononanoate.

### Example 7

### Production of Skin Lotion

Components shown in Table 2 were uniformly dissolved at 60° C.-70° C., then cooled to 30° C., to prepare a skin lotion.

TABLE 2

| | |
|---|---|
| | Blending quantity |
| Component name | (% by mass) |

| | | |
|---|---|---|
| 1 | Esterified compound of Example 3 | 20.00 |
| 2 | Citric acid | 0.01 |
| 3 | Sodium citrate | 0.09 |
| 4 | Pentylene glycol | 3.00 |
| 5 | Purified water | Remainder |

### Production of Skin Lotion

Components shown in Table 3 were uniformly dissolved at 60° C.-70° C., then cooled to 30° C., to prepare a skin lotion.

TABLE 3

| | | Blending quantity |
|---|---|---|
| Component name | | (% by mass) |
| 1 | Esterified compound of Example 1 | 30.00 |
| 2 | Citric acid | 0.01 |
| 3 | Sodium citrate | 0.09 |
| 4 | Mixture of phenoxyethanol, methylparaben, | 0.40 |
| | ethylparaben, butylparaben, propylparaben | |
| | and isobutylparaben | |
| 5 | Purified water | Remainder |

### Production of Skin Lotion

Components shown in Table 4 were uniformly dissolved at 60° C.-70° C., then cooled to 30° C., to prepare a skin lotion.

TABLE 4

| | |
|---|---|
| | Blending quantity |
| Component name | (% by mass) |

| | | |
|---|---|---|
| 1 | Esterified compound of Example 2 | 15.00 |
| 2 | Disodium hydrogenphosphate | 0.02 |
| 3 | Sodium dihydrogen phosphate | 0.08 |
| 4 | Pentylene glycol | 3.00 |
| 5 | Aloe extract | Appropriate quantity |
| 6 | Purified water | Remainder |

### Example 10

### Production of Skin Lotion

Components shown in Table 5 were uniformly dissolved at 60° C.-70° C., then cooled to 30° C., to prepare a skin lotion.

TABLE 5

| | |
|---|---|
| | Blending quantity |
| Component name | (% by mass) |

| | | |
|---|---|---|
| 1 | Esterified compound of Example 5 | 15.00 |
| 2 | Citric acid | 0.01 |
| 3 | Sodium citrate | 0.09 |
| 4 | Pentylene glycol | 3.00 |
| 5 | Dipotassium Glycyrrhizinate | 0.04 |
| 6 | Purified water | Remainder |

### Example 11

### Production of Skin Lotion

Components shown in Table 6 were uniformly dissolved at 60° C.-70° C., then cooled to 30° C., to prepare a skin lotion.

TABLE 6

| | |
|---|---|
| | Blending quantity |
| Component name | (% by mass) |

| | | |
|---|---|---|
| 1 | Esterified compound of Example 1 | 15.00 |
| 2 | EDTA-4Na | 0.05 |
| 3 | Pentylene glycol | 3.00 |
| 4 | glycerin | 2.00 |
| 5 | Fragrance | Appropriate quantity |
| 6 | Purified water | Remainder |

### Example 12

### Production of Skin Lotion

Components shown in Table 7 were uniformly dissolved at 60° C.-70° C., then cooled to 30° C., to prepare a skin lotion.

TABLE 7

| | |
|---|---|
| | Blending quantity |
| Component name | (% by mass) |

| | | |
|---|---|---|
| 1 | Esterified compound of Example 4 | 15.00 |
| 2 | Citric acid | 0.01 |
| 3 | Sodium citrate | 0.09 |
| 4 | Pentylene glycol | 3.00 |
| 5 | Butylene Glycol | 3.00 |
| 6 | Purified water | Remainder |

### Example 13

### Production of Skin Cream (O/W Type Cream)

Composition A and composition B shown in Table 8 were individually and uniformly dissolved at 75° C.-80° C. Then composition B was added to composition A while stirring, and the mixture was emulsified using a homo-mixer. The mixture was subsequently cooled to 30° C. while stirring, to prepare an O/W type skin cream.

TABLE 8

| | | | Blending quantity |
|---|---|---|---|
| Component name | | | (% by mass) |
| A | 1 | Squalane | 10.00 |
| | 2 | Hydrogenated Rapeseed Alcohol | 1.00 |
| | 3 | Behenyl alcohol | 2.00 |
| | 4 | (Polyglyceryl-2 isostearate/dimer | 2.00 |
| | | dilinoleate) copolymer | |
| | 5 | Polyglyceryl-10 stearate | 1.50 |
| | 6 | Pentylene glycol | 3.00 |
| B | 7 | Esterified compound of Example 1 | 10.00 |
| | 8 | Citric acid | 0.01 |
| | 9 | Sodium citrate | 0.09 |
| | 10 | Xanthan gum | 0.08 |
| | 11 | (Ammonium acryloyldimethyltaurate/ | 0.22 |
| | | beheneth-25 methacrylate) copolymer | |
| | 12 | Purified water | Remainder |

### Example 14

### Production of Cleansing Oil

Components shown in Table 9 were uniformly dissolved at 80° C., then cooled to 30° C., to prepare a cleansing oil.

TABLE 9

| | |
|---|---|
| | Blending quantity |
| Component name | (% by mass) |

| | | |
|---|---|---|
| 1 | Esterified compound of Example 4 | 45.00 |
| 2 | Neopentyl glycol diisononanoate | Remainder |
| 3 | PEG-20 glyceryl diisostearate | 15.00 |
| 4 | Polyglyceryl-2 triisostearate | 10.00 |
| 5 | Tocopherol | Appropriate quantity |

### Example 15

### Production of Cleansing Oil

Components shown in Table 10 were uniformly dissolved at 80° C., then cooled to 30° C., to prepare a cleansing oil.

TABLE 10

| | |
|---|---|
| | Blending quantity |
| Component name | (% by mass) |

| | | |
|---|---|---|
| 1 | Esterified compound of Example 1 | 30.00 |
| 2 | Isononyl isononanoate | Remainder |
| 3 | PEG-20 glyceryl diisostearate | 10.00 |
| 4 | PEG-8 diisostearate | 8.00 |
| 5 | Polyglyceryl-2 isostearate | 5.00 |
| 6 | Polyglyceryl-2 triisostearate | 5.00 |
| 7 | Tocopherol | Appropriate quantity |

### Example 16

### Production of Cleansing Oil

Components shown in Table 11 were uniformly dissolved at 80° C., then cooled to 30° C., to prepare a cleansing oil.

TABLE 11

| | |
|---|---|
| | Blending quantity |
| Component name | (% by mass) |

| | | |
|---|---|---|
| 1 | Esterified compound of Example 5 | 30.00 |
| 2 | Neopentyl glycol diethylhexanoate | Remainder |
| 3 | PEG-30 glyceryl trioleate | 12.00 |
| 4 | PEG-8 diisostearate | 8.00 |
| 5 | Polyglyceryl-2 isostearate | 4.00 |
| 6 | Polyglyceryl-2 triisostearate | 5.00 |
| 7 | Tocopherol | Appropriate quantity |

### Example 17

### Production of Shampoo

In accordance with the standard process except that the esterified compound of Example 6 and components 4 and 5 shown in Table 12 were preliminarily dissolved by heating, all the components were uniformly dissolved at 75° C.-80° C., then cooled to 30° C. to prepare a shampoo.

TABLE 12

| | | Blending quantity |
|---|---|---|
| Component name | | (% by mass) |
| 1 | Esterified compound of Example 6 | 5.00 |
| 2 | Polyquaternium-10 | 1.00 |
| 3 | Purified water | Remainder |
| 4 | Polyglyceryl-10 isostearate | 1.50 |
| 5 | Polyglyceryl-2 laurate | 1.00 |
| 6 | Lauryl betaine (30% aqueous solution) | 10.00 |
| 7 | Cocamide DEA | 4.00 |
| 8 | Lauryldimethylamine oxide | 1.00 |
| 9 | Ceteareth-60 myristyl glycol | 2.00 |
| 10 | Sodium cocoyl methyl taurine (30% aqueous | 27.00 |
| | solution) | |
| 11 | Sodium laureth sulfate (30% aqueous solution) | 23.00 |
| 12 | Glycol distearate | 2.00 |
| 13 | Pentylene glycol | 3.00 |
| 14 | Stearamide propyl dimethylamine | 0.30 |
| 15 | Citric acid (10% aqueous solution) | 3.50 |

### Example 18

### Production of Rinse-Off Hair Treatment

Composition A and composition B shown in Table 13 were individually and uniformly dissolved at 75° C.-80° C. Then composition B was added to composition A while stirring, and the mixture was emulsified using a homo-mixer. The mixture was cooled to 30° C. while stirring, to prepare a rinse-off hair treatment.

TABLE 13

| | | | Blending quantity |
|---|---|---|---|
| Component name | | | (% by mass) |
| A | 1 | Esterified compound of Example 3 | 8.00 |
| | 2 | Polyglyceryl-10 laurate | 0.50 |
| | 3 | Polyglyceryl-2 triisostearate | 2.00 |
| | 4 | Glyceryl stearate | 1.00 |
| | 5 | Cetyl palmitate | 0.50 |
| | 6 | Cetearyl alcohol | 4.40 |
| | 7 | Behenyl alcohol | 1.10 |
| | 8 | Behen trimonium chloride | 2.70 |
| | 9 | Quaternium-18 | 0.40 |
| | 10 | Dipropylene Glycol | 2.00 |
| | 11 | Steareth-6 | 1.50 |
| | 12 | Steareth-3 | 1.50 |
| | 13 | Dimethicone | 1.00 |
| B | 14 | Hydroxypropylmethyl cellulose | 0.30 |
| | 15 | Pentylene glycol | 3.00 |
| | 16 | Polyquaternium-7 | 1.00 |
| | 17 | Purified water | Remainder |

### Example 19

### Production of Aqueous Lip Gloss

Components 2-6 and 8 shown in Table 14 were uniformly dissolved at 90° C.-105° C. Meanwhile, component 1 and component 7 were uniformly mixed. Subsequently all these components were uniformly mixed at 80° C., then cooled to 30° C. to prepare an aqueous lip gloss.

TABLE 14

| | |
|---|---|
| | Blending quantity |
| Component name | (% by mass) |

| | | |
|---|---|---|
| 1 | Esterified compound of Example 5 | 10.00 |
| 2 | (Polyglyceryl-2 isostearate/dimer | 40.00 |
| | dilinoleate) copolymer | |
| 3 | Diisostearyl malate | 15.00 |
| 4 | Polyglyceryl-2 triisostearate | 8.00 |
| 5 | Pentaerythrityl tetraisostearate | Remainder |
| 6 | Dextrin (palmitate/ethylhexanoate) | 4.00 |
| 7 | Purified water | 5.00 |
| 8 | Antiseptic | Appropriate quantity |

### Example 20

### Production of Aqueous Lip Gloss

Components 2-8 and 10 shown in Table 15 were uniformly dissolved at 90° C.-105° C. Meanwhile, component 1 and component 9 were uniformly mixed. Subsequently all these components were uniformly mixed at 80° C., then cooled to 30° C. to prepare an aqueous lip gloss.

TABLE 15

| | |
|---|---|
| | Blending quantity |
| Component name | (% by mass) |

| | | |
|---|---|---|
| 1 | Esterified compound of Example 6 | 10.00 |
| 2 | (Polyglyceryl-2 isostearate/dimer | 20.00 |
| | dilinoleate) copolymer | |
| 3 | Hydrogenated polyisobutene | 20.00 |
| 4 | Diisostearyl malate | 12.00 |
| 5 | Polyglyceryl-2 diisostearate | 10.00 |
| 6 | Isotridecyl isononanoate | Remainder |
| 7 | Dextrin palmitate | 3.50 |
| 8 | Di-C-20-40 alkyl dimer dilinoleate | 1.00 |
| 9 | Purified water | 2.00 |
| 10 | Antiseptic | Appropriate quantity |

### Example 21

### Production of O/W Type Emulsified Foundation

Components 4-8 shown in Table 16 were preliminarily dispersed in components 1, 2 and 3 using a homo-mixer. Composition A and composition B were individually and uniformly dissolved at 75° C.-80° C. Subsequently, composition A was gradually added to composition B while stirring, and the mixture was emulsified by a homo-mixer. It was subsequently cooled to 30° C. while stirring, to prepare an O/W type emulsified foundation.

TABLE 16

| | | | Blending quantity |
|---|---|---|---|
| Component name | | | (% by mass) |
| A | 1 | Neopentyl glycol diisononanoate | 10.00 |
| | 2 | Squalane | 5.00 |
| | 3 | Cyclomethicone | 3.00 |
| | 4 | Talc | 5.00 |
| | 5 | Titanium oxide | 7.00 |
| | 6 | Iron oxide (red oxide of iron) | 1.00 |
| | 7 | Iron oxide (yellow oxide of iron) | 3.50 |
| | 8 | Iron oxide (black oxide of iron) | 0.50 |
| | 9 | Hydrogenated Rapeseed Oil Alcohol | 1.00 |
| | 10 | Behenyl alcohol | 2.00 |
| | 11 | (Polyglyceryl-2 isostearate/dimer | 2.00 |
| | | dilinoleate) copolymer | |
| | 12 | Polyglyceryl-10 stearate | 1.50 |
| | 13 | Pentylene glycol | 3.00 |
| B | 14 | Esterified compound of Example 4 | 5.00 |
| | 15 | Citric acid | 0.01 |
| | 16 | Sodium citrate | 0.09 |
| | 17 | Xanthan gum | 0.08 |
| | 18 | (Ammonium acryloyldimethyltaurate/ | 0.22 |
| | | beheneth-25 methacrylate) copolymer | |
| | 19 | Purified water | Remainder |

### Example 22

### Hair Mist

Components 1-5 and 7 shown in Table 17 were uniformly dissolved at 80° C. then cooled to 50° C. Component 6 was subsequently added, and the mixture was further cooled to 30° C. to prepare a hair mist.

TABLE 17

| | |
|---|---|
| | Blending quantity |
| Component name | (% by mass) |

| | | |
|---|---|---|
| 1 | Esterified compound of Example 1 | 5.00 |
| 2 | Citric acid | 0.01 |
| 3 | Sodium citrate | 0.09 |
| 4 | Behenyl trimethyl ammonium chloride | 0.50 |
| 5 | Pentylene glycol | 3.00 |
| 6 | Fragrance | 0.04 |
| 7 | Purified water | Remainder |

### Example 23

### Hair Mist

Components 1-6 and 8 shown in Table 18 were uniformly dissolved at 80° C., then cooled to 50° C. Component 7 was subsequently added, and the mixture was further cooled to 30° C. to prepare a hair mist.

TABLE 18

| | |
|---|---|
| | Blending quantity |
| Component name | (% by mass) |

| | | |
|---|---|---|
| 1 | Esterified compound of Example 2 | 5.00 |
| 2 | (Polyglyceryl-2 isostearate/dimer | 2.00 |
| | dilinoleate) copolymer | |
| 3 | Citric acid | 0.01 |
| 4 | Sodium citrate | 0.09 |
| 5 | Behenyl trimethyl ammonium chloride | 0.50 |
| 6 | Pentylene glycol | 3.00 |
| 7 | Fragrance | 0.04 |
| 8 | Purified water | Remainder |

### Example 24

### Bath Agent

Components 1-5 and 7-9 shown in Table 19 were uniformly dissolved at 60° C., then cooled to 50° C. Component 6 was subsequently added, and the mixture was further cooled to 30° C., to prepare a bath agent.

TABLE 19

| | | Blending quantity |
|---|---|---|
| | Component name | (% by mass) |
| 1 | Esterified compound of Example 5 | 8.00 |
| 2 | Citric acid | 0.01 |
| 3 | Sodium citrate | 0.09 |
| 4 | Jojoba oil | 3.00 |
| 5 | Polygryceryl-10 laurate | 0.50 |
| 6 | Pentylene glycol | 2.50 |
| 7 | Tocopherol | 0.01 |
| 8 | Fragrance | 0.04 |
| 9 | Purified water | Remainder |

### BRIEF DESCRIPTION OF DRAWINGS

FIG. 1: Diagram showing an IR chart of dicarbitol succinate.

FIG. 2: Diagram showing a GC/MS chart of dicarbitol succinate.

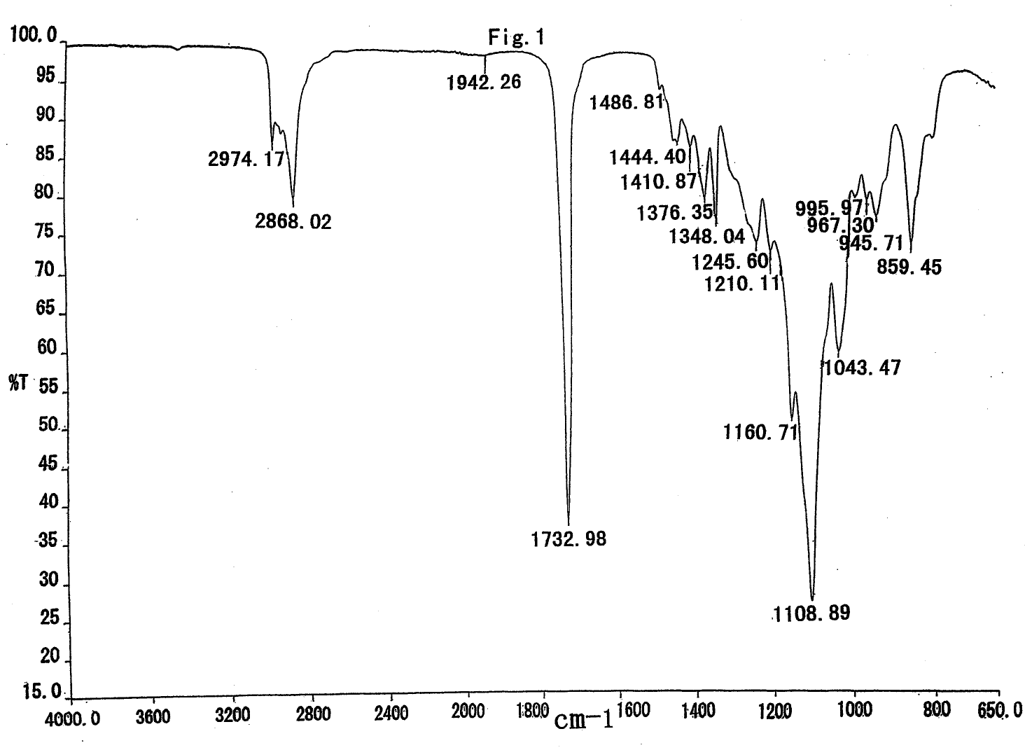

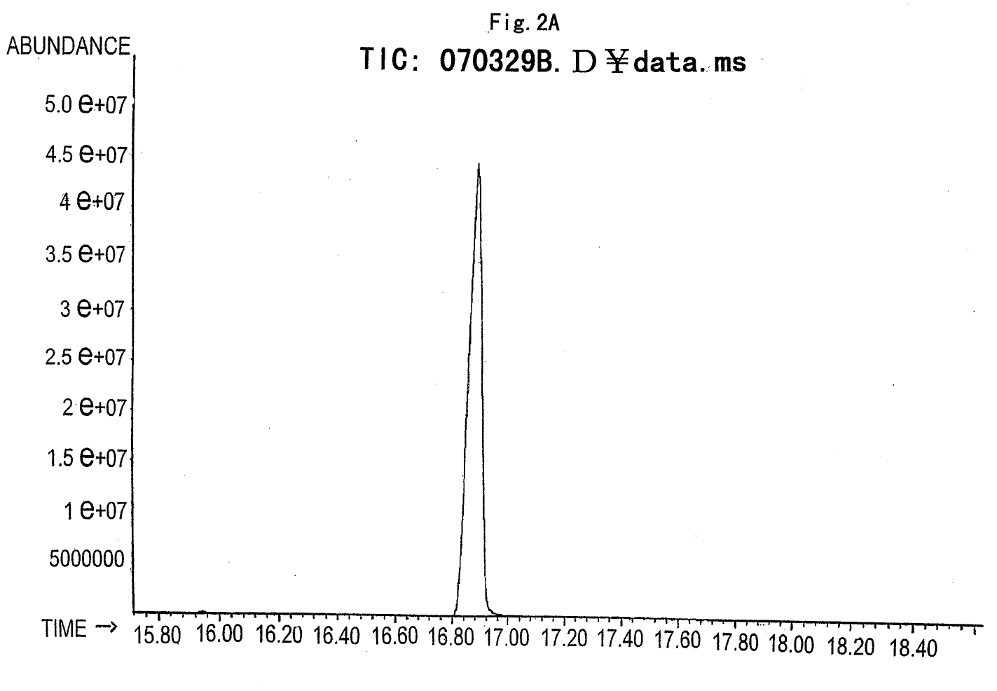

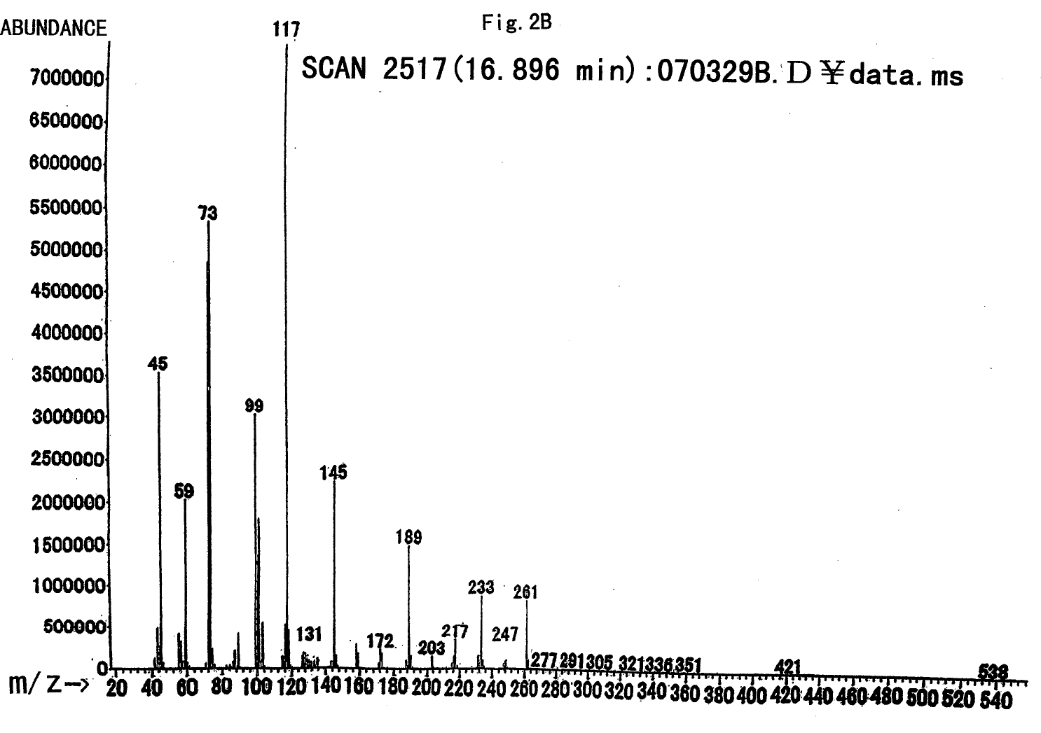

1. An oil agent comprising one or more dibasic acid ester compounds of general formula I: 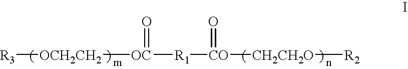
wherein R₁ is an alkylene group having a carbon number of 2-4, which may be mono- or poly substituted, R₂ and R₃ are, independently of each other, an alkyl group having a carbon number of 1-4, which may be mono- or poly substituted, n and m are, independently of each other, an integer of 0-4 with m+n≧1 (provided that the cases wherein both of R₂ and R₃ are an ethyl group and both of n and m are 1 are excluded).

2. The oil agent according to claim 1, wherein a poly- (or mono-)ethylene glycol monoether that constitutes the ester compound is one or two substances selected from the group consisting of diethylene glycol monoethyl ether, triethylene glycol monoethyl ether, diethylene glycol monopropyl ether, triethylene glycol monopropyl ether, diethylene glycol monoisopropyl ether and triethylene glycol monoisopropyl ether.

3. The oil agent according to claim 1, wherein the dibasic acid is malic acid, succinic acid, tartaric acid or adipic acid.

4. The oil agent according to claim 1, wherein the dibasic acid is succinic acid.

5. The oil agent according to claim 1, wherein the solubility in water is 5% by mass or more at 20° C.

6. The oil agent according to claim 1, wherein the viscosity is 5-200 (mPa·s) at 25° C.

7. A lubricating agent comprising the oil agent according to claim 1.

8. A moisturizing agent comprising the oil agent according to claim 1.

9. An external preparation comprising the oil agent according to claim 1.

10. The external preparation according to claim 9, wherein it is a cosmetic material.
